(19) United States
(12) Patent Application Publication
(10) Pub. No.: US 2014/0308361 A1
(43) Pub. Date: Oct. 16, 2014

(54) COMPOSITION FOR MANAGEMENT OF PERIODONTAL DISEASES

(71) Applicants: KING ABDULLAH INTERNATIONAL MEDICAL RESEARCH CENTER, Riyadh (SA); KING SAUD BIN ABDULAZIZ UNIVERSITY FOR HEALTH SCIENCES, Riyadh (SA); NATIONAL GURARD HEALTH AFFAIRS, Riyadh (SA)

(72) Inventors: NOHA MOHAMED ZAKI RAYAD, Taif (SA); Ibrahim Maghrabi, Taif (SA); Mohamed Mostafa Hafez Mahmoud, Taif (SA)

(21) Appl. No.: 14/318,208

(22) Filed: Jun. 27, 2014

Related U.S. Application Data
(62) Division of application No. 13/769,800, filed on Feb. 18, 2013.

(51) Int. Cl. A61K 9/06; A61K 31/5383; A61K 31/24; A61K 9/00

(52) U.S. Cl. CPC A61K 9/06; A61K 9/0063; A61K 31/5383; A61K 31/24
USPC 424/499; 514/230.2; 514/534; 424/501

(57) ABSTRACT

The composition for management of periodontal diseases includes a gel matrix having a polymer system and a plurality of microspheres dispersed in the polymer system. The polymer system contains about one-half a dose of medicament, while the micro spheres contain the remainder. Upon administration of the composition into the periodontal cavity, the medicament in the polymer system provides an initial therapeutic benefit, while the remainder of the medication is released over time via degradation of the microspheres. This biphasic pattern of medicament delivery provides increased efficacy of the medicament through sustained delivery of the same.

### BACKGROUND OF THE INVENTION

1. Field of the Invention

The present invention relates to periodontal disease treatments, and particularly to a composition for management of periodontal diseases that provides maximal effective delivery of medicament into the periodontal pocket.

2. Description of the Related Art

Periodontal disease is an infection caused by bacteria in the biofilm or dental plaque that forms on oral surfaces. The disease causes deterioration of the teeth and gums in the oral cavity and typically manifests as lesions in various states of progression. Usually, the disease begins as gingivitis, an inflammation of the gums, which can lead to periodontitis, a condition in which the patient exhibits progressive loss of the alveolar bone around the teeth. Left untreated, the teeth will loosen and the patient will eventually lose the teeth.

The oral cavity is home to a host of bacteria, at least 500 or so identified bacterium, and the body is in constant struggle combating these bacteria. In general, waste products from these bacteria cause destruction of tissue and halitosis. Due to the complex etiology of these bacteria, it has been difficult to identify a particular pathogen for periodontal disease. However, recent advances in molecular biological techniques have enabled easier identification of periodontopathic bacteria.

Regular brushing and flossing are common measures that reduce risks of periodontal disease. However, many factors are involved with the onset of the disease. Studies have shown that while advanced age is a common factor, other factors, such as genetics, tobacco use, gender, and diabetes mellitus, are also found to be culpable.

Several treatments exist to counter periodontal diseases. One common form of treatment involves rinsing subgingival pockets with a solution of hydrogen peroxide, typically in concentrations of 1%-3%. The hydrogen peroxide acts as an antimicrobial agent. Another treatment involves an antibiotic, such as doxycycline, orally administered to the patient. A still further treatment involves injection of medication in the periodontal cavity. In the latter case, the efficacy of the medication is somewhat diminished due to hindered transmucosal delivery having an effect on absorption and the absorption rate.

In light of the above, it would be a benefit in the art of periodontal disease treatment to provide a composition that insures effective delivery of drugs to counter the effects of periodontal disease. Thus, a composition for management of periodontal diseases solving the aforementioned problems is desired.

### SUMMARY OF THE INVENTION

The composition for management of periodontal diseases includes a polymer system forming a gel matrix, and a plurality of microspheres dispersed in the polymer system. The polymer system contains about one-half the dose of medicament, while the microspheres contain the remainder. Upon administration of the composition into the periodontal cavity, the medicament in the polymer system provides an initial therapeutic benefit, while the remainder of the medication is released over time via degradation of the microspheres. This biphasic pattern of medicament delivery provides increased efficacy of the medicament through sustained delivery of the same.

These and other features of the present invention will become readily apparent upon further review of the following specification and drawings.

### BRIEF DESCRIPTION OF THE DRAWINGS

FIG. 1 is an environmental, perspective view of a composition for management of periodontal diseases being administered to a patient.

FIG. 2 is a chart comparing the antibacterial effect of the composition for management of periodontal diseases compared to a conventional solution over time.

FIG. 3 is a chart comparing the probing depth (PD) and clinical attachment level (CAL) reductions in patients treated with the composition for management of periodontal diseases and the conventional solution.

Similar reference characters denote corresponding features consistently throughout the attached drawings.

### DETAILED DESCRIPTION OF THE PREFERRED EMBODIMENTS

The composition for management of periodontal diseases provides sustained therapeutic levels of medication delivery for treating periodontal disease. The composition includes a polymer system forming a gel matrix containing about one-half of a drug or medicament dose, and a plurality of microspheres dispersed in the polymer system, the microspheres containing the remainder of the prescribed drug dose.

The polymer system is configured to deliver rapid therapeutic levels of the drug into the gingival crevicular fluid (GCF). The polymer system can include chitosan provided in about 0.5-5% by weight concentration, and/or poloxamer members at about 16-25% by weight concentration alone or in a mixture of the above. Chitosan has proven to exhibit antibiofilm and antibacterial effect against periodontopathic bacteria, especially Porphyromonas gingivalis. Poloxamer has also been shown to demonstrate antadherence effect against bacteria. Hence, the poloxamer can counteract plaque formation.

The microspheres contain the remainder of the medicament and provide a time-release mechanism for delivering the rest of the dose over a predetermined period. This facilitates a controlled and sustained release of the medicament, which greatly enhances efficacy of the treatment. The microspheres are constructed from ethylcellulose (EC), poly(lactide-co-glycolide) polymers (PLGA), polycaprolactone (PCL), and the like that exhibit high biocompatibility and biodegradation. Preferably, the microspheres have a particle size ranging from about 50-800 μm. The drug to polymer ratio is preferably about 1:2 or 1:4.

The drug or medicament for the composition can be an antibiotic or a local anesthetic. An exemplary antibiotic may be ofloxacin at about 0.1-1% concentration by weight. An exemplary anesthetic may be mebeverine HCl at about 10-50% concentration by weight. Although mebeverine HCl is more commonly used as an antispasmodic, especially for colon spasms, it has been found that the medicament demonstrates successful local anesthetic effect.

As briefly mentioned above, it is preferable that the polymers used in preparation of the composition exhibit a high degree of biocompatibility and biodegradation. The former is self-explanatory, while the latter insures a proper rate of deterioration for delivering the medicament. The above characteristics insure the composition remains in the periodontal cavity and administers the prescribed amount of medicament in a biphasic pattern for longer-lasting clinical improvement, i.e., the polymer system delivers the first half for an initial therapeutic benefit and the microspheres deliver the rest over time to extend that benefit. Moreover, the rheological properties are readily adjustable to facilitate ease of injection via a syringe and filling of the periodontal cavity.

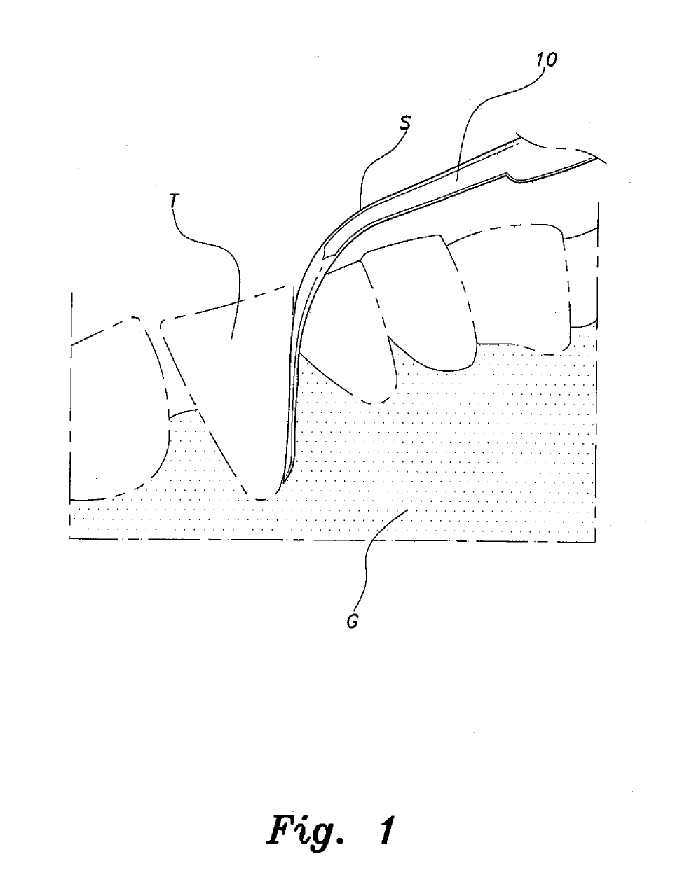

FIG. 1 shows an example of administering the composition 10. As shown, the dentist uses a syringe S filled with the composition 10 prepared in the manner described above. The syringe S is placed near the injection site between the teeth T and the gum G. The dentist injects the composition 10 into the periodontal cavity below the gum line.

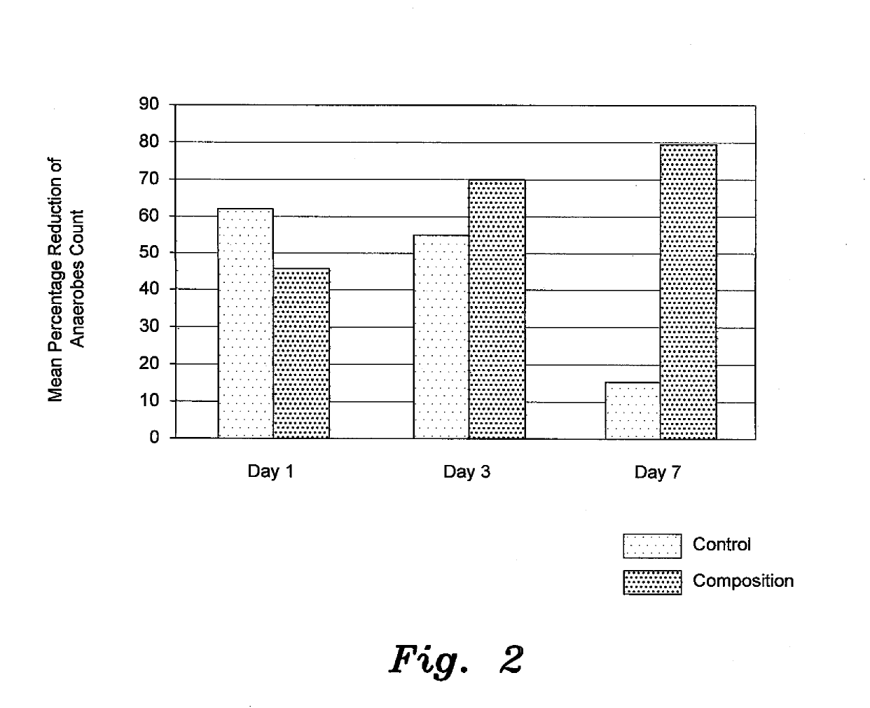

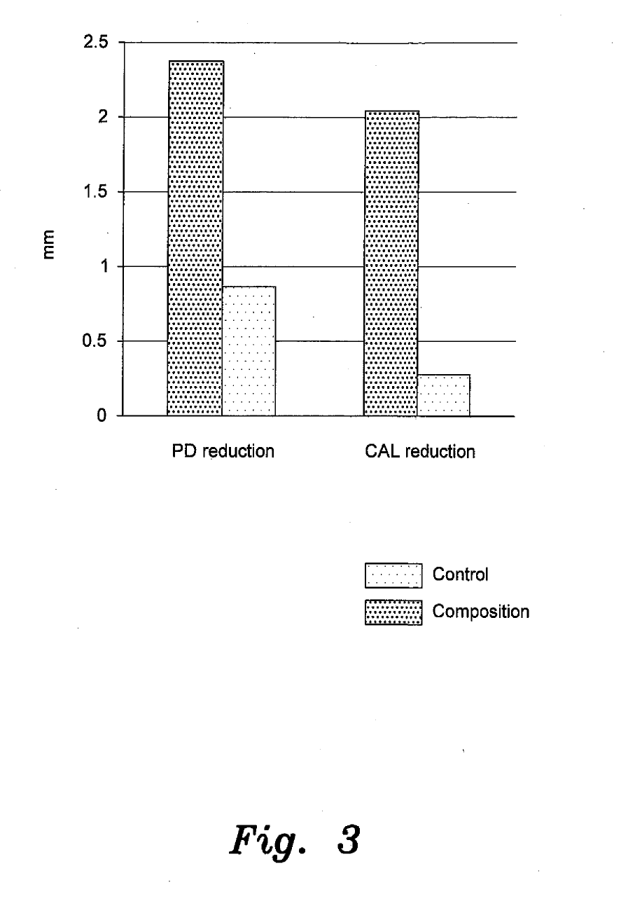

The above procedure has been used in tests, and the charts shown in FIGS. 2 and 3 demonstrate the effectiveness of the composition over injection of conventional medicament (indicated as “Control” in the drawings). FIG. 2 shows a comparison of the mean percentage reduction of anaerobes count between the composition 10 and the conventional modes of administering the medicament over a week, or seven days. Most bacteria responsible for periodontal disease are anaerobic. Thus, a greater percentage in anaerobe count reduction correlates to greater efficacy of the treatment. It can be seen from FIG. 2 that while the mean percentage of reduction of anaerobes was initially low, about 47% for the present composition compared to about 62% from the control, the therapeutic benefit of the composition was maintained and continuously increased throughout the week. At the end of the testing period, the composition exhibited about 78% anaerobes count reduction, while the control was substantially lower at about 15%. This suggests that while the initial or short term therapeutic benefit of the conventional medicament may be effective, the long term benefit of the composition far surpassed that of the conventional medicament.

FIG. 3 shows the comparison of probing depth (PD) and the clinical attachment level (CAL) between the composition and the control. PD is defined as the distance from the gingival margin to the bottom of the cavity or pocket, measured in millimeters (mm). PD serves as an indicator of the severity of the periodontal disease. CAL is defined as the distance from the cement-enamel junction (CEJ) to the bottom of the cavity, also measured in mm. CAL serves as the primary measure of the efficacy of the treatment. In both parameters, high reduction of either measurement indicates a greater degree of therapeutic treatment benefit. The results of FIG. 3 are derived from measurements taken from the beginning and end of the seven day period. It can be seen from FIG. 3 that the composition demonstrated a substantial reduction in both PD and CAL, compared to the control. The PD reduction was about 2.4 mm and 0.8 mm respectively. This suggests that the composition was about 300% more effective in healing the gum compared to the control. The CAL reduction was about 2.1 mm and 0.25 mm, respectively. This suggests that the efficacy of the composition was much greater than the efficacy from the control.

Thus, it can be seen that the composition 10 for managing periodontal disease provides improved therapeutic treatment of the disease. The gel matrix facilitates a biphasic delivery pattern for the medicament, which greatly increases the efficacy through long-term, sustained administration of the medicament.

It is to be understood that the present invention is not limited to the embodiments described above, but encompasses any and all embodiments within the scope of the following claims.

1. A delivery system for management of periodontal diseases, comprising:
a polymer system forming a gel matrix adapted for carrying about one-half of a dose of a medicament for treating periodontal disease, the gel matrix providing rapid release of the medicament to achieve therapeutic levels, wherein said polymer system comprises 0.5-5% by weight of chitosan and 16-25% by weight poloxamer members; and
a plurality of microspheres suspended and dispersed in the gel matrix, the microspheres containing the remainder of the dose, the microspheres providing gradual time release of the medicament to maintain the therapeutic levels of medicament for a sustained period of time, said microspheres comprise a polymer selected from the group consisting of ethylcellulose, poly(lactide-co-glycolide) polymer, and polycaprolactone polymer, and said microspheres having a particle size ranging from about 50-800 μm;
whereby the gel matrix provides adjustable rheological properties to facilitate ease of injection via syringe, and whereby the gel matrix provides antibiofilm and anti-bacterial effects, as well as anti-adherence of periodontopathic bacteria.

2. (canceled)

3. (canceled)

4. (canceled)

5. (canceled)

6. (canceled)

7. (canceled)

8. The delivery system according to claim 1, wherein said microspheres are constructed from biodegradable and biocompatible polymers.

9. (canceled)

10. (canceled)

11. (canceled)

12. A composition for management of periodontal diseases, comprising:
a polymer system forming a gel matrix, wherein said polymer system comprises 0.5-5% by weight of chitosan and 16-25% by weight poloxamer members;
a plurality of microspheres suspended and dispersed in the gel matrix, the microspheres including a polymer selected from the group consisting of ethylcellulose, poly(lactide-co-glycolide) polymer, and polycaprolactone polymer, and having a particle size ranging from about 50-800 μm;
an effective amount of an active ingredient for the management of periodontal disease, about one-half of the effective amount being dispersed in the gel matrix for rapid release of therapeutic levels of the active ingredient, the microspheres containing the remainder of the effective amount and providing gradual time release of the active ingredient to maintain the therapeutic levels of medicament for a sustained period of time
whereby the gel matrix provides adjustable rheological properties to facilitate ease of injection via syringe, and whereby the gel matrix provides antibiofilm and anti-bacterial effects, as well as anti-adherence of periodontopathic bacteria.

13. (canceled)

14. (canceled)

15. The composition for management of periodontal diseases according to claim 14, wherein said active ingredient comprises an antibiotic.

16. The composition for management of periodontal diseases according to claim 14, wherein said active ingredient comprises ofloxacin.

17. The composition for management of periodontal diseases according to claim 14, wherein said active ingredient comprises an anesthetic.

18. The composition for management of periodontal diseases according to claim 14, wherein said active ingredient comprises mebeverine HCl.

19. (canceled)

20. (canceled)
